(19) United States
(12) Plant Patent Application Publication
(10) Pub. No.: US 2019/0110385 P1
(43) Pub. Date: Apr. 11, 2019

(54) Strawberry plant named 'EMS 1371'

(50) ;

(71) Applicant: Enrique Masiá Serrador, Lepe (ES)

(72) Inventor: Enrique Masiá Serrador, Lepe (ES)

(21) Appl. No.: 15/932,970

(22) Filed: May 31, 2018

Related U.S. Application Data
(60) Provisional application No. 62/604,134, filed on Jun. 22, 2017.

(51) Int. Cl. A01H 6/74

(52) U.S. Cl. PLT/208

(57) ABSTRACT

A new day-neutral strawberry plant named ‘A13-71’ is disclosed, with exceptional yield and flavor characteristics.

### CROSS-REFERENCE TO RELATED APPLICATIONS

This application claims priority of co-pending provisional application Ser. No. 62/604,134 as filed on Jun. 22, 2017.

### STATEMENT REGARDING FEDERALLY SPONSORED RESEARCH OR DEVELOPMENT

None

Genus and species: Fragaris x ananassa Duch.

Variety denomination: ‘A13-71’.

### BACKGROUND OF THE INVENTION

‘A13-71’ is a product of a controlled breeding program carried out by the inventor in the Andalucia region of Spain. ‘A13-71’ was one of several seedlings resulting from a controlled cross. A single plant of ‘A13-71’was initially selected based on observations of its fruiting characteristics. Later, ‘A13-71’ was asexually propagated by rooting stolons and was expanded to 30 plants which were planted in replicated trials in the Andalucia region of Spain. The plants were observed and evaluated, and the next year they were expanded for further observation and evaluation. The breeders of ‘A13-71’ has sold the variety commercially under the Trademark name Cleopatra WB or Cleopatra Circle R Brand. Throughout several generations of asexual propagation, ‘A13-71’ has been observed to retain its distinctive characteristics and remain true to type.

### BRIEF DESCRIPTION OF THE PHOTOGRAPH

FIG. 1 illustrates mature strawberries of ‘A13-71’;

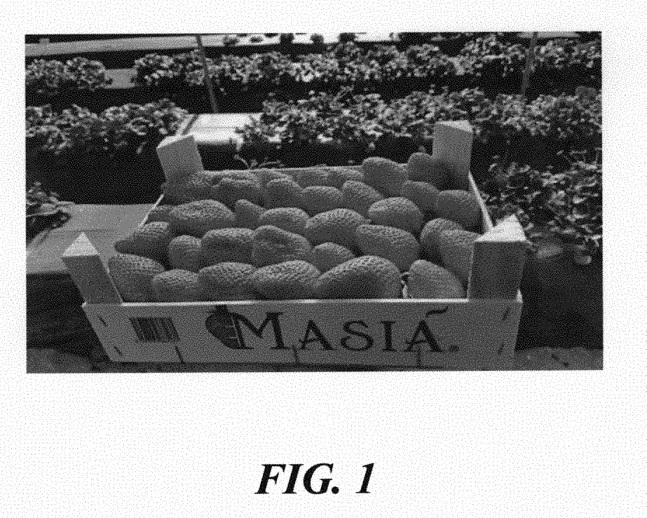

FIG. 2 illustrates individual whole berries and cross sections of whole berries of mature strawberries of ‘A13-71’;

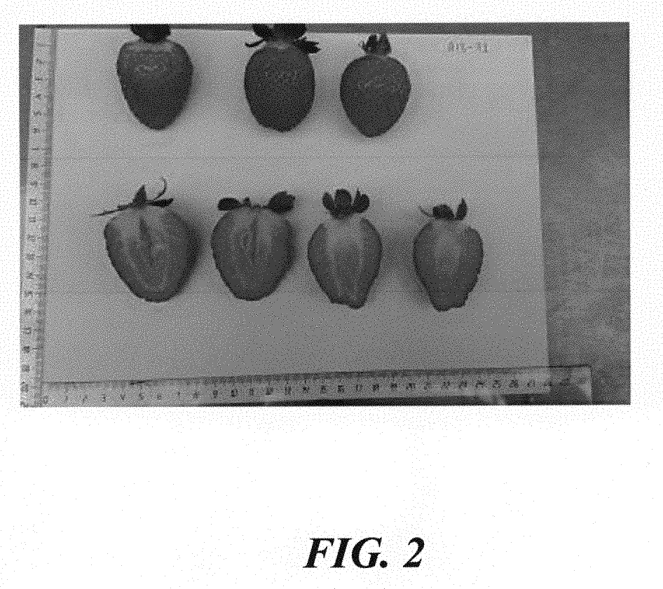

FIG. 3 illustrates mature plants of ‘A13-71’ growing in a raised bench system; and

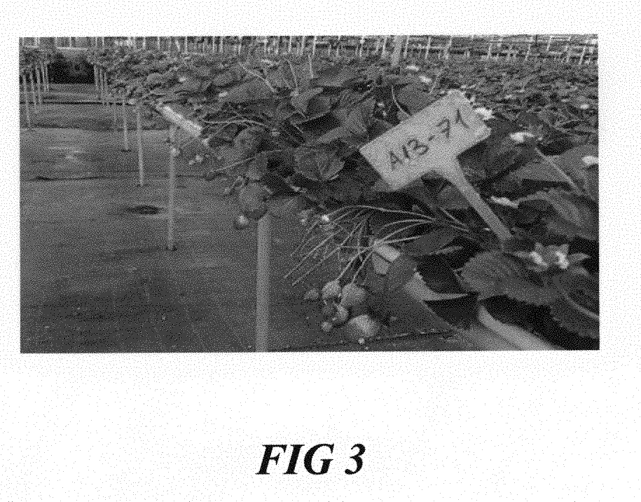

FIG. 4 illustrates immature plants ‘A13-71’ growing in a traditional raised bed with black plastic mulch.

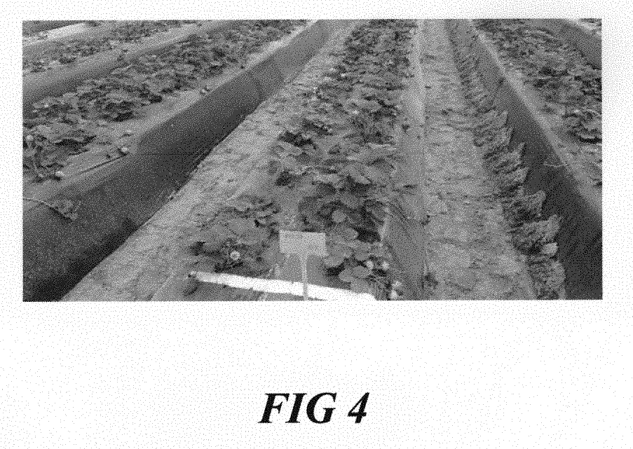

The colors of these illustrations may vary with lighting conditions and, therefore, color characteristics of this new variety should be determined with reference to the observations described herein, rather than from these illustrations alone.

### DETAILED BOTANICAL DESCRIPTION

The following description is based on observations made during the 2016 and 2017 growing seasons at in the Andalucia region of Spain. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant or any group of plants, of the new variety may vary from the stated average.
General description:
   Ploidy.—Octoploid.
   Blooming period.—Approximately January through May in Huelva, Spain.
   Plant type.—Herbaceous fruit producing perennial.
   Plant habit.—Upright, semi-compact with dense canopy.
   Cold hardiness.—Not tested in areas where temperatures of 32° F. occur.
   Root description.—Fibrous, 10 days to initiate roots, 8 weeks to produce a rooted plant, White NN155C in color.
   Propagation.—Rooting of stolons.
   Growth rate.—Vigorous.
   Stem.—Acaulescent.
   Stolon description.—Yellow Green 114B to 145A in color.
Foliage description:
   Leaf division.—Three leaflets.
   Leaf arrangement.—Basal.
   Leaf attachment.—Petiolate.
   Leaflet shape.—Broadly ovate to rounded.
   Leaflet margins.—Serrate, slightly pointed to slightly rounded.
   Leaflet base.—Asymmetrically oblique and rounded.
   Leaflet apex.—Round.
   Leaflet venation.—Pinnate, coloration matched leaflet color.
   Leaflet color.—Upper surface Dark Green 137A, lower surface Grayed-green 191A, no variegation present on either surface.
   Petiole.—Round in shape, strong in strength, Yellow Green 144B and 145A in color.
   Petiolules.—Round in shape, Yellow Green 145A in color.
   Stipule.—Purple Red 55 AB and C.
Flower description:
   Inflorescence.—Truss.
   Flower initiation and expression conditions.—Temperature and day-length dependent.
   Time of flowering (50% of plants at first flower).—Early to mid-season.
   Flower fragrance.—Slight.
   Sepals.—Upper-surface is Dark Green 137A; lower surface is Grayed-green 191A.
   Sepal position.—Mixed arrangement relative to the fruit.
   Petals.—Round in shape, obtuse base and apex, slightly overlapping, entire margins, Grey 155C.
   Peduncle.—Yellow Green 145A, strong in strength.
   Pedicel.—Yellow Green 145A, strong in strength.
   Pistils.—Average of 145, average of 1.1 mm in length, steeply dome shaped, Yellow Green 151B and 151C.
   Stigma.—Yellow Green 154A and 154B.
   Style.—Yellow Green 154A and 154B.
   Stamens.—Average of 26, Moderate Yellow 162B, pollen is moderate in quantity and Vivid Greenish Yellow 2A in color.
   Bracts.—Observed on the majority of the flower trusses from early developmental stage, which progresses into a typical single leaflet as the truss matures and fruit develops with characteristics similar to leaflets.
Fruit description:
   Shape.—Primarily conical to cordate with broad shoulders, shape is similar for primary, secondary, and tertiary fruit.
   Season of harvest.—January through May in Huelva, Spain.
   Time of ripening (50% of plants with first ripe fruit).—Early.
   Time of bearing.—Day-Neutral, It will flower independent of day length.
   Size.—Large to medium.
   Surface.—Smooth and glossy.
   Glossiness.—Even and very high.
   External color (skin).—Red 44A and 44B, color is retained throughout the cropping season and holds up well to high seasonal temperatures.
   Internal color.—Orange Red 32A.
   Evenness of color of skin.—Even.
   Acidity.—Low.
   Sweetness.—High.
   Firmness.—Skin is very firm (resistant to bruising), flesh is moderately firm.
   Juiciness.—Moderate.
   Aroma.—High.
   Weight.—Average of 15 to 20 g per berry and 600 to 1000 g per plant.
   Shelf life.—An average of 8 to 10 days.
   Achene.—Brilliant Greenish Yellow 1B on the shady side, Vivid Reddish Orange 34B on the sunny side.
   Achene number.—An average of 250 per berry.

What is claimed is:

1. A new and distinct strawberry plant as shown and described herein.
